(19) United States
(12) Patent Application Publication
(10) Pub. No.: US 2008/0269342 A1
(43) Pub. Date: Oct. 30, 2008

(54) Pleuromutilin Derivatives and Its Use

(76) Inventors: David H. Igo, Durham, NC (US); Beth A. Norton, Durham, NC (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/093,729

(22) PCT Filed: Nov. 18, 2006
(86) PCT No.: PCT/US06/61066

§ 371 Date: May 15, 2008

Related U.S. Application Data
(60) Provisional application No. 60/738,308, filed on Nov. 18, 2005.

(51) Int. Cl. A61K 31/185; C07C 237/24

(52) U.S. Cl. 514/616; 564/157

(57) ABSTRACT

The invention is directed to the L-tartrate salt of trans-3-aminocyclobutyl (1S,2R,3S,4S,6R,7R,8,14R)-4-ethenyl-3-hydroxy-2,4,7,14-tetramethyl-9-oxotricyclo[5.4.3.01,8]tetradec-6-yl imidodicarbonate (Compound IA.) Compound IA is useful for the treatment of a variety of diseases and conditions, such as respiratory tract and skin and skin structure infections. Accordingly, the invention is further directed to pharmaceutical compositions comprising Compound IA. The invention is still further directed to methods of treating respiratory tract and skin and skin structure infections using Compound IA or a pharmaceutical composition comprising Compound IA.

### FIELD OF THE INVENTION

The invention is directed to the L-tartrate salt of trans-4-aminocyclohexyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate depicted herein as Compound IA and its use in the treatment of respiratory tract and skin and skin structure infections.

### BACKGROUND OF THE INVENTION

International Application No. PCT/EP01/11603, published as International Publication No. WO 02/30929, discloses certain pleuromutilin derivatives useful as antibacterial agents. Specifically, WO 02/30929 discloses C-14 oxycarbonyl carbamate pleuromutilin derivatives according to Formula IA or Formula IB therein.

One such C-14 oxycarbonyl carbamate pleuromutilin derivative encompassed within Formula IA of WO 02/30929 is trans-3-aminocyclobutyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate (“Compound I”). While Compound I is encompassed within Formula IA of WO 02/30929, it is not specifically disclosed in the specification or claims. Compound I is represented by the following structure:

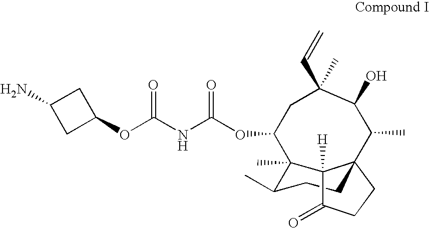

In addition, WO 02/30929 discloses that the compounds disclosed therein that contain a basic group “may be in the form of a free base or an acid addition salt.” Pharmaceutically acceptable salts, such as though described by Berge et al. (J. Pharm Sci., 1977, 66, 1-19) are indicated as preferred salts. Hydrochloride, maleate, and methanesulfonate are specifically mentioned.

Compound I has recently been identified as a particularly useful compound because it has demonstrated good in vitro and in vivo activity against representative Gram-positive and Gram-negative pathogens associated with respiratory tract and skin and skin structure infections including isolates resistant to existing classes of antimicrobials.

In view of the good in vitro and in vivo activity exhibited by Compound I against representative Gram-positive and Gram-negative pathogens associated with respiratory tract and skin and skin structure infections there is a need for a form of Compound I suitable for pharmaceutical development.

### SUMMARY OF THE INVENTION

The invention is directed to the L-tartrate salt of trans-3-aminocyclobutyl (1S,2R,3S,4S,6R,7R,8R, 14R)-4-ethenyl-3-hydroxy-2,4,7,14-tetramethyl-9-oxotricyclo[5.4.3.01,8]tetradec-6-yl imidodicarbonate depicted herein as Compound IA. Compound IA is useful for the treatment of a variety of diseases and conditions, such as respiratory tract and skin and skin structure infections. Accordingly, the invention is further directed to pharmaceutical compositions comprising Compound IA. The invention is still further directed to methods of treating respiratory tract and skin and skin structure infections using Compound IA or a pharmaceutical composition comprising Compound IA.

### BRIEF DESCRIPTION OF THE FIGURES

FIG. 1 is an x-ray powder diffractogram of Compound IA.

### DETAILED DESCRIPTION OF THE INVENTION

In describing the invention, chemical elements are identified in accordance with the Periodic Table of the Elements. Abbreviations and symbols utilized herein are in accordance with the common usage of such abbreviations and symbols by those skilled in the chemical and biological arts. For example, the following abbreviations are used herein:

“g” is an abbreviation for grams

“mL” is an abbreviation for milliliters

“C” is an abbreviation for degrees Celsius

“DMF” is an abbreviation for the solvent N,N-dimethylformamide

“DSC” is an abbreviation for Differential Scanning Calorimetry

“vol” or “vols” refers to is an abbreviation for volume or volumes, respectively, and refers to the amount of solvent used relative the weight of a starting material. One volume of solvent is defined as 1 mL of solvent for every 1 g of starting material.

“eq” is an abbreviation for molar equivalents

“THF” is an abbreviation for the solvent tetrahydrofuran

“L” is an abbreviation for liters

“N” is an abbreviation for Normal and refers to the number of equivalents of reagent per liter of solution.

“mmos” is an abbreviation for millimole or millimolar

“mol” is an abbreviation for mole or molar

“LOD” is an abbreviation for Loss on Drying

“HPLC” is an abbreviation for High Pressure Liquid Chromatography

“NMR” is an abbreviation of Nuclear Magnetic Resonance

“TLC” is an abbreviation for Thin Layer Chromatography

“LCMS” is an abbreviation for Liquid Chromatography/Mass Spectroscopy

“KF” is an abbreviation for Karl Fischer water determination

“JLR” is an abbreviation for Jacketed Lab Reactor

“TG” and “TGA” are abbreviations for ThermoGravinmetric Analysis

“IPA” is an abbreviation for isopropanol, and is also known as 2-propanol

“NMP” is an abbreviation for N-methylpyrrolidinone

“ppm” is an abbreviation for parts per million

### Compound IA

The invention is directed to the L-tartrate salt of trans-3-aminocyclobutyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate depicted below as Compound IA.

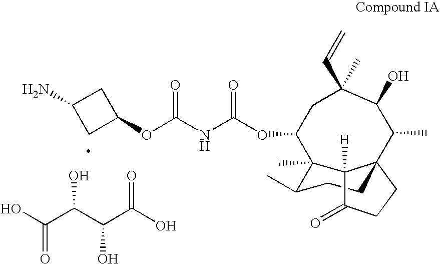

Surprisingly, it has been found that Compound IA has advantageous physical properties that make it particularly well suited for pharmaceutical development.

In the solid state, Compound IA can exist in crystalline, semi-crystalline and amorphous structures, as well as mixtures thereof. The skilled artisan will appreciate that pharmaceutically-acceptable solvates of Compound IA may be formed wherein solvent molecules are incorporated into the solid-state structure during preparation. Solvates may involve non-aqueous solvents such as ethanol, isopropanol (also referred to as 2-propanol), n-propanol (also referred to as 1-propanol), DMSO, acetic acid, ethanolamine, acetonitrile, and ethyl acetate, or they may involve water as the solvent that is incorporated into the solid-state structure. In addition, the solvent content of Compound IA can vary in response to environment and upon storage, for example, water may displace another solvent over time depending on relative humidity and temperature.

Solvates wherein water is the solvent that is incorporated into the solid-state structure are typically referred to as “hydrates.” Solvates wherein more than one solvent is incorporated into the solid-state structure are typically referred to as “mixed solvates”. Solvates include “stoichiometric solvates” as well as compositions containing variable amounts of solvent (referred to as “non-stoichiometric solvates”). Stoichiometric solvates wherein water is the solvent that is incorporated into the solid-state structure are typically referred to as “stoichiometric hydrates”, and non-stoichiometric solvates wherein water is the solvent that is incorporated into the solid-state structure are typically referred to as “non-stoichiometric hydrates”. The invention includes both stoichiometric and non-stoichiometric solvates.

In addition, solid-state structures of Compound IA, including solvates thereof, may contain solvent molecules, which are not incorporated into the solid-state structure. For example, solvent molecules may become trapped within larger particles upon isolation. In addition, solvent molecules may be retained on the surface of the crystals. The invention includes such solid-state structures of Compound IA.

The skilled artisan will further appreciate that Compound IA, including solvates thereof, may exhibit polymorphism (i.e. the capacity to occur in different crystalline packing arrangements). Different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different IR spectra, solid-state NMR spectra, and X-ray powder diffraction patterns, which may be used for identification. Polymorphs may also exhibit different melting points, which may be used for identification. The skilled artisan will appreciate that different polymorphs may be produced, for example, by changing or adjusting the reaction conditions or reagents, used in making the compound. For example, changes in temperature, pressure, or solvent may result in the production of different polymorphs. In addition, one polymorph may spontaneously convert to another polymorph under certain conditions.

### REPRESENTATIVE EMBODIMENTS

In one embodiment, the invention is directed to Compound IA in the solid state. In one embodiment, the invention is directed to Compound IA in crystalline form. In another embodiment, the invention is directed to Compound IA in semi-crystalline form. In another embodiment, the invention is directed to Compound IA in amorphous form.

In another embodiment, the invention is directed to substantially pure Compound IA. As used herein, the term “substantially pure” when used is reference to Compound IA refers to a product which is greater than about 90% pure. Preferably, “substantially pure” refers to a product which is greater than about 95% pure, and more preferably greater than about 97% pure. This means the product does not contain any more than about 10%, 5% or 3% respectively of any other compound.

In another embodiment, the invention is directed to a non-stoichiometric hydrate of Compound IA containing from about 2% to about 7% water. In another embodiment, the invention is directed to a non-stoichiometric hydrate of Compound IA containing from about 2% to about 6% water. In another embodiment, the invention is directed to a non-stoichiometric hydrate of Compound IA containing from about 4% to about 6% water.

In one embodiment, the solid-state structure of Compound IA is characterized by an x-ray powder diffraction (XRPD) pattern having characteristic peaks at the following positions: 6.7±0.2 (°2θ), 10.0±0.2 (°2θ), 11.7±0.2 (°2θ), 13.2±0.2 (° 2θ), 13.7±0.2 (°2θ), 14.2±0.2 (° 2θ), 20.4±0.2 (° 2θ), and 23.5±0.2 (° 2θ). In addition to these XRPD peaks, several additional peaks present in the patterns may vary with solvent and water content. Accordingly, in another embodiment, the invention is directed to Compound IA in the solid state wherein the solid-state structure of Compound IA is characterized by an XRPD pattern having at least one characteristic peak selected from characteristic peaks at the following positions: 6.7±0.2 (°2θ), 10.0±0.2 (°2θ), 11.7±0.2 (°2θ), 13.2±0.2 (°2θ), 13.7 z 0.2 (°2θ), 14.2±0.2 (°2θ), 20.4±0.2 (° 2θ), and 23.5±0.2 (° 2θ). In another embodiment, the invention is directed to Compound IA in the solid state wherein the solid-state structure of Compound IA is characterized by an XRPD pattern having at least two characteristic peaks selected from characteristic peaks at the following positions: 6.7±0.2 (°2θ), 10.01±0.2 (°2θ), 11.7±0.2 (°2θ), 13.2±0.2 (°2θ), 13.7±0.2 (°2θ), 14.2±0.2 (° 2θ), 20.4±0.2 (° 2θ), and 23.5±0.2 (° 2θ). In another embodiment, the invention is directed to Compound IA in the solid state wherein the solid-state structure of Compound IA is characterized by an XRPD pattern having at least three characteristic peaks selected from characteristic peaks at the following positions: 6.7±0.2 (°2θ), 10.01±0.2 (°2θ), 11.7±0.2 (°2θ), 13.2±0.2 (°2θ), 13.7±0.2 (°2θ), 14.2±0.2 (°2θ), 20.4±0.2 (°2θ), and 23.5±0.2 (° 2θ). In another embodiment, the invention is directed to Compound IA in the solid state wherein the solid-state structure of Compound IA is characterized by an XRPD pattern having at least four characteristic peaks selected from characteristic peaks at the following positions: 6.7±0.2 (°2θ), 10.0±0.2 (° 2θ), 11.7±0.2 (°2θ), 13.2±0.2 (°2θ), 13.7±0.2 (° 2θ), 14.2±0.2 (°2θ), 20.4±0.2 (° 2θ), and 23.5±0.2 (° 2θ). In another embodiment, the invention is directed to Compound IA in the solid state wherein the solid-state structure of Compound IA is characterized by an XRPD pattern having at least five characteristic peaks selected from characteristic peaks at the following positions: 6.7±0.2 (°2θ), 10.0±0.2 (°2θ), 11.7±0.2 (°2θ), 13.2±0.2 (°2θ), 13.7±0.2 (°2θ), 14.2±0.2 (°2θ), 20.4±0.2 (° 2θ), and 23.5±0.2 (° 2θ). In another embodiment, the invention is directed to Compound IA in the solid state wherein the solid-state structure of Compound IA is characterized by an XRPD pattern having at least six characteristic peaks selected from characteristic peaks at the following positions: 6.7±0.2 (° 2θ), 10.0±0.2 (° 2θ), 11.7±0.2 (° 2θ), 13.2±0.2 (° 2θ), 13.7±0.2 (° 2θ), 14.2±0.2 (° 2θ), 20.4±0.2 (° 2θ), and 23.5±0.2 (° 2θ). In another embodiment, the invention is directed to Compound IA in the solid state wherein the solid-state structure of Compound IA is characterized by an XRPD pattern having at least seven characteristic peaks selected from characteristic peaks at the following positions: 6.7±0.2 (°2θ), 10.0±0.2 (°2θ), 11.7±0.2 (° 2θ), 13.2±0.2 (° 2θ), 13.7±0.2 (°2θ), 14.2±0.2 (° 2θ), 20.4±0.2 (° 2θ), and 23.5±0.2 (° 2θ). In another embodiment, the invention is directed to Compound IA in the solid state wherein the solid-state structure of Compound IA is characterized by an XRPD pattern having characteristic peaks at the following positions: 6.7±0.2 (° 2θ), 10.0±0.2 (°2θ), 11.7±0.2 (° 2θ), 13.2±0.2 (° 2θ), 13.7±0.2 (° 2θ), 14.2±0.2 (° 2θ), 20.4±0.2 (° 2θ), and 23.5±0.2 (° 2θ).

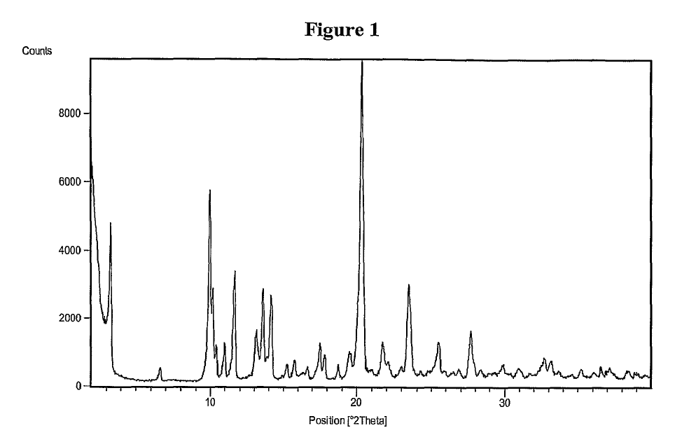

In another embodiment, the invention is directed to Compound IA in the solid state wherein the solid-state structure of Compound IA is characterized by substantially the same XRPD pattern as depicted in FIG. 1.

The XRPD data described herein was acquired using a Philips X'Pert Pro powder X-ray diffractometer. Samples were gently flattened onto a zero-background silicon holder. A continuous 2θ scan range of 2° to 40° was used with a CuKα radiation source and a generator power of 40 kV and 40 mA. A 2θ step size of 0.0167 degrees/step with a step time of 10.16 seconds was used. Samples were rotated at 25 rpm and all experiments were performed at room temperature. Characteristic XRPD peak positions are reported in units of angular position (20) with a precision of +/−0.1°, which is caused by instrumental variability and calibration.

The location (° 2θ values) of these peaks was obtained from an XRPD pattern expressed in terms of 2-theta angles and obtained with a diffractometer using copper Kα-radiation. The XRPD patterns provided herein are expressed in terms of 2-theta angles and obtained with a diffractometer using copper Kα-radiation. It will be understood by those skilled in the art that an XRPD pattern will be considered to be substantially the same as a given XRPD pattern if the difference in peak positions of the XRPD patterns are not more than +0.2 (° 2θ).

In order to maintain the crystallinity of Compound IA when in crystalline form, the compound should not be exposed to a temperature above about 95° C.

### Compound Preparation

Compound IA is generally prepared from pleuromutilin or from mutilin. Pleuromutilin may be produced by the fermentation of microorganisms such as Clitopilus species, Octojuga species and Psathyrella species using methods known to those skilled in the art. The pleuromutilin is then isolated from the fermentation broth with organic solvent. Pleuromutilin may be converted to mutilin by alkaline hydrolysis. Such methods are well known in the art.

For example, Compound IA may be prepared from “Intermediate 1” (depicted below). The preparation of Intermediate I is described below in Examples 1, 2, and 3. Other starting materials and reagents are commercially available or are made from commercially available starting materials using known methods.

### EXAMPLES

The following preparation examples are not intended to limit the scope of the present invention, but rather to provide guidance to the skilled artisan to prepare the compounds of the invention.

### Example 1

### Preparation of Intermediate 1

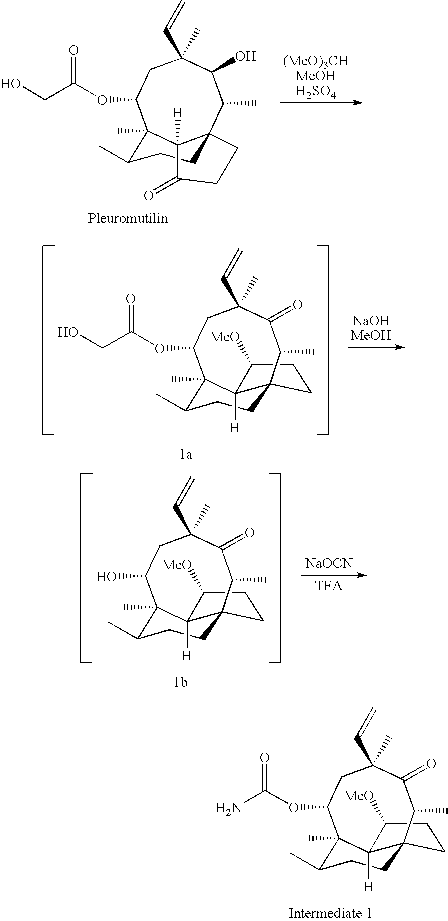

To a reaction vessel under nitrogen atmosphere were charged pleuromutilin (59.2 grams), methanol (240 mL) and trimethyl orthoformate (95 mL). The mixture was cooled to 0° C. Concentrated sulfuric acid (18 mL) was added slowly to keep the reaction temperature below 10° C. After addition, the reaction mixture was heated to 30° C. After 3 hours at 30° C. and 14 hours at 18° C., the reaction was deemed complete by HPLC analysis. The crude product in the reaction mixture was used in next reaction directly.

1a in the reaction mixture was cooled to −10° C. Water (70 mL) was added slowly to keep the internal temperature below 15° C. An aqueous solution of sodium hydroxide (135 mL, 50% w/w) was charged slowly to keep the internal temperature below 15° C. The reaction was then heated to 65° C. After 30 minutes at 65° C. the reaction was complete based on HPLC analysis. The reaction was cooled to ˜40° C. Methanol was distilled out under reduced pressure. Water (300 mL) and toluene (350 mL) were added to the mixture. The mixture was heated to ˜65° C. and was stirred for 10 minutes. After settling for 30 minutes, the aqueous layer was separated. The aqueous layer was extracted with toluene (200 ml). The organic layers were combined and distilled under reduced pressure to a final volume of 300 mL. The crude product in toluene was used directly in next reaction.

To the product from above in toluene was added more toluene (350 mL) at ambient temperature. Sodium cyanate (27.4 grams) was added with stirring. Trifluoroacetic acid (29 mL) was slowly added over 0.5 hour. The mixture was stirred for 14 hours at ambient temperature. No starting material was detected in the reaction mixture by HPLC analysis. Water (360 mL) was added to the reaction with stirring. The layers were separated and the aqueous layer was discarded. Toluene was distilled under reduced pressure until a final volume of 100 mL. Heptane (300 mL) was added. The mixture was stirred at 65° C. for 30 minutes then cooled to 0° C. and stirred for one hour. The resulting slurry was filtered and washed twice with cold heptane (80 mL). The crude product was dried at 65-70° C. under vacuum to give 42.1 grams of Intermediate 1. Yield: 71%.

### Example 2

### Preparation of Intermediate I

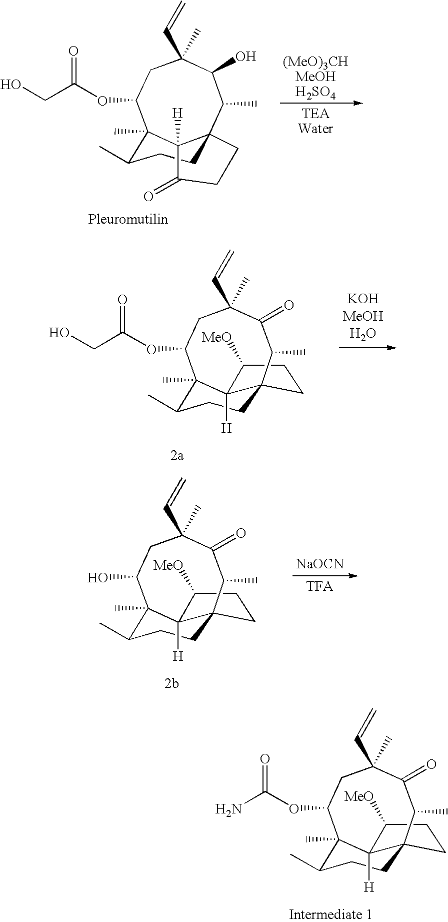

To a reaction vessel under nitrogen atmosphere were charged pleuromutilin (20.0 grams), methanol (80 mL) and trimethyl orthoformate (32 mL). The mixture was cooled to 0° C. Concentrated sulfuric acid (6 mL) was added slowly to keep the reaction temperature below 10° C. After addition, the reaction mixture was heated to 30° C. After 5 hours at 30° C. and 14 hours at 18° C., the reaction was deemed completed by HPLC analysis. The reaction mixture was cooled to ˜10° C. Triethylamine (32 mL) was added slowly to keep the internal temperature below 30° C. Water (110 mL) was added to the reaction with vigorous stirring. The mixture was stirred at ˜20° C. for 4 hours. The crude product was filtered and washed with water (60 mL) twice. The wet solid was dried at 50° C. under vacuum to give 16.0 grams of product. Yield: 77%.

To a flask were charged methanol (80 mL) and water (10 mL). Potassium hydroxide (5.7 g) was added. The mixture was stirred for 5 minutes to a solution. 2a (20.0 g) was added to the mixture. The reaction mixture was heated to 65° C. and stirred for 1 hour. The reaction was deemed complete by HPLC analysis and the mixture was cooled to ˜25° C. and slowly transferred into a larger flask containing water (100 mL) and 2b seed (50 mg) with vigorous stirring. The resulted slurry was cooled to ˜5° C. and stirred for 1 hour. The crude 2b was filtered and washed with water (50 mL) twice. The wet product was dried at ˜65° C. for 24 hours to give 15.3 grams of solid. Yield: 90%.

To a flask were charged toluene (180 mL), 2b (20.0 g) and sodium cyanate with stirring. Trifluoroacetic acid (10 mL) was slowly added over 1 hour. The mixture was stirred for 16 hours at ambient temperature after which no 2b was detected by HPLC analysis. Water (100 mL) was added to reaction with stirring and the layers were separated. The aqueous layer was discarded and the toluene layer was concentrated under reduced pressure to a final volume of ˜30 mL. Heptane (100 mL) was added and the mixture was stirred at 65° C. for 30 minutes. The mixture was cooled to 0° C. and stirred for 1 hour. The resulted slurry was filtered and washed with cold heptane (20 mL, ˜0° C.) twice. The crude product was dried at 65° C. under vacuum to give 19.1 grams of Intermediate 1. Yield: 85%.

### Example 3

### Preparation of Intermediate 1

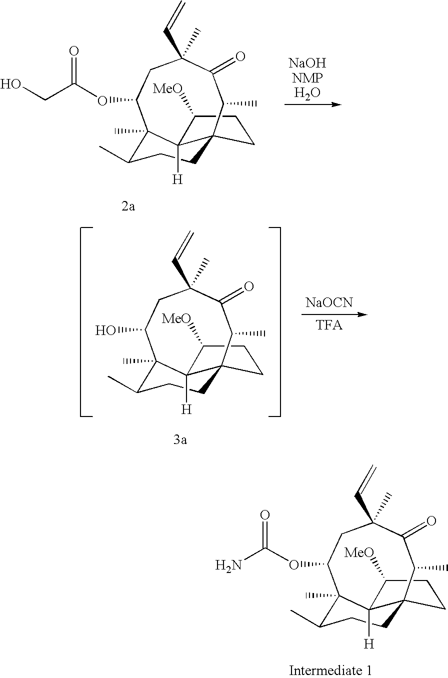

To a flask were charged N-methylpyrrolidone (24 mL), 2a solid (from Example 2) (12.0 grams), and water (10 mL). Sodium hydroxide aqueous solution (20 mL, 50% w/w) was added. The reaction mixture was heated to 70° C. and stirred for 1 hour. Toluene (120 mL) was added to the mixture, stirred for 30 minutes and the layers were separated. The toluene layer was washed with water (30 mL) and concentrated under vacuum to ˜100 mL final volume. The crude product in toluene was used directly in the next reaction.

To 3a in toluene was added sodium cyanate. Trifluoroacetic acid (5 mL) was slowly added over 1 hour. The mixture was stirred for ˜15 hours at ambient temperature until no 3a was detected by HPLC analysis. Water (30 mL) was added to reaction with stirring, the layers were separated, and the aqueous layer was discarded. Toluene was distilled under reduced pressure until ˜10 mL remained. Heptane (50 mL) was added and the mixture was stirred at 65° C. for 30 minutes. The mixture was cooled to 0° C. and stirred for one hour. The resulting slurry was filtered and was washed twice with cold heptane (15 mL each, ˜0° C.). The crude product was dried at 65° C. under vacuum to give 9.5 grams of Intermediate 1. Yield: 82%.

### Example 4

### Preparation of Compound IA

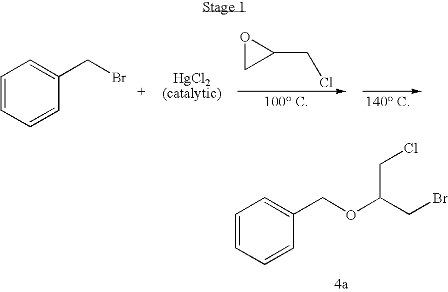

To a solution of mercury chloride (5.3 g,) in benzyl bromide (2.31 kg,) at 100° C. or reflux was slowly added epichlorohydrin (1.25 kg) over forty minutes. The reaction mixture was then heated to an internal temperature about ˜135° C. for ˜3 hours, cooled to room temperature overnight and heated for an additional ˜12 h at ˜135-150° C. The mixture was cooled to ambient temperature and left overnight. The mixture was then purified via reduced pressure distillation. A yield of 70% 1-Bromo-2-O-benzyl-3-chloropropane (4a, 2.51 kg) was obtained.

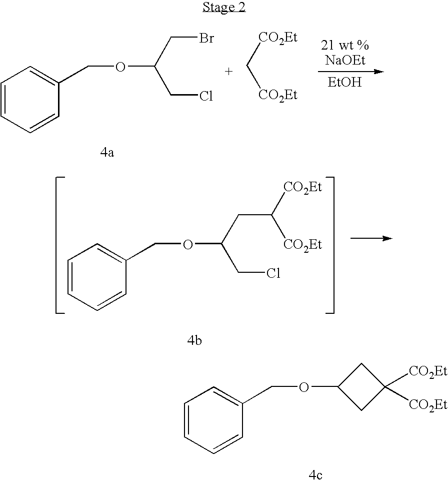

To a solution of 4a (80 g) and diethyl malonate (121.7 g, 2.5 equiv) in EtOH (160 mL) was slowly charged NaOEt (21 wt % in EtOH) (284 mL, 2.5 equiv) through addition funnel. The mixture was heated to reflux (˜80° C. internal temperature) then stirred for additional 3 hours before sampling and concluding that 4a was consumed based on HPLC results. The mixture was cooled to ˜35° C. and filtered through filter paper. The filtrate was concentrated by distillation until ˜420 mL of distilled solvent was collected. The mixture was heated to ˜125° C. and stirred for 2 hours before sampling and concluding that 4b was consumed based on HPLC results. The mixture was cooled to room temperature. Water (160 mL) and ethyl acetate (320 mL) were charged. The mixture was stirred and two layers were separated. The organic layer was washed with water (80 mL). The organic layer was concentrated under reduced pressure to dryness. The product was dried under vacuum to obtain crude 4c, 125.1 g.

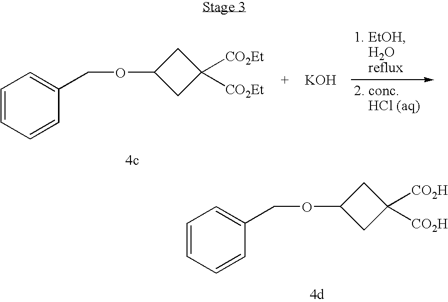

A KOH solution was prepared by adding KOH (2.02 kg, 5 equiv) to water (2.55 L). A 20 L jacketed laboratory reactor was charged with crude 4c (1.7 kg,) and EtOH (6.8 L). The KOH solution was charged in 2 portions which caused the internal temperature to rise to 56° C. The mixture was heated over ˜40 minutes until brought to reflux (˜79° C. internal temperature) then stirred for and additional 30 minutes before sampling and concluding that 4c was consumed based on HPLC results. The mixture was cooled slightly then concentrated under reduced pressure until ˜5.2 L of solution remained in the reactor. While continuing to cool the reactor, the contents were diluted with water (5.1 L). When the temperature of the solution reached ˜16° C., concentrated HCl (aqueous) was slowly added in portions until the aqueous layer pH was adjusted to 2.5-3 (a total of 2.2 L of concentrated HCl was used). Methyl t-butyl ether (8.5 L) was charged. The mixture was stirred and the layers were separated. The organic layer was washed with water (1.7 L). The organic layer was held at ˜20° C. overnight, then concentrated under reduced pressure until ˜2.8 L remained in the reactor. Toluene (8.5 L) was charged and the mixture was concentrated under reduced pressure until ˜6.8 L of distilled solvent was collected. Toluene (6.8 L) was charged and the mixture was heated to ˜90° C. over 50 minutes, then cooled back to 16° C. over 50 minutes. The solid was filtered and rinsed with cyclohexane (1.7 L). The solid was dried tinder vacuum at 50° C. for ˜2 days and 1.04 kg of dried product 4d was obtained.

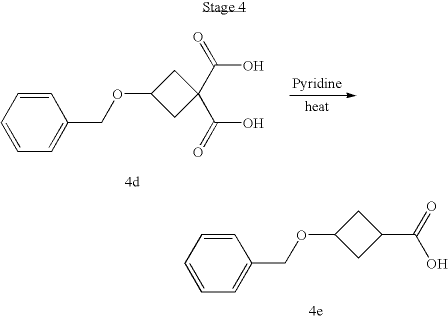

4d (783.35 g) was charged to a 3 L round bottom flask. Pyridine (783 mL,.) was added. The solution was heated to ˜117° C. for 8-12 hours. The reaction was deemed complete when HPLC monitoring indicated that <2% of 4d remained. The solution was concentrated under vacuum on a rotary evaporator until no additional distillate could be seen. After holding the residue overnight, toluene (4.7 L) was added, followed by slow addition of an HCl solution (1.0 N, 3.13 L) at such a rate that the temperature was maintained <30° C. during the addition. The mixture was stirred for 10 minutes. The two layers were separated and the aqueous layer was extracted with 2.35 L of toluene. The combined organic layers were washed with 783 mL of brine and the layers were again separated. The organic layer was concentrated until 2.35 L of solution remained. Further concentration under vacuum provided an oil (619 g). The yield was estimated to be ˜77% based on a weight/weight assay using HPLC.

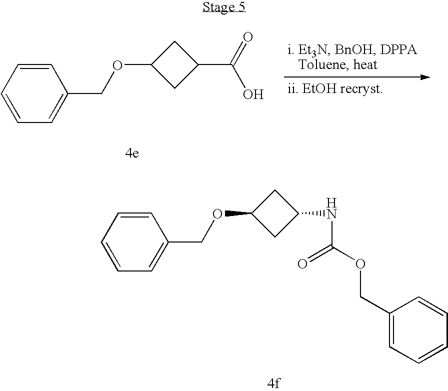

A solution of 4e in toluene (assumed 3.6 kg, 17.5 mol), triethylamine (3.5 kg, 34.9 mol), and benzylalcohol (1.9 kg, 17.5 mol) in total amount of toluene (36 L) was prepared then heated to an internal temperature between 70 and 80° C. Diphenylphosphorylazide (4.9 kg, 18.0 mol) was slowly added over 35 minutes while maintaining temperature between 70 and 80° C. The vessel containing the diphenylphosphorylazide was rinsed with 1 L toluene and added to reactor. Once the addition was complete, the contents were held for ˜15 minutes at ˜80° C., then the reaction mixture was heated to an internal temperature ˜100° C. and stirred for about 11.5 hours. The reaction mixture was cooled to ˜20° C., and held overnight. The reaction mixture was partially concentrated by vacuum distillation to ˜15 L. Ethyl acetate (EtOAc, 40 L) and 0.25 N aqueous sodium hydroxide solution (18 kg) were added. The layers were separated. The organic layer was washed with 0.25 N NaOH (22.4 kg). EtOAc was removed via vacuum distillation to a minimum stirrable volume (˜18 L). Ethanol (200 proof, 18 L) was added and residual EtOAc was removed via vacuum distillation to ˜18 L. The mixture was heated to 75° C. to dissolve all solids (˜15 min), then cooled to between 30 and 40° C. The mixture was seeded with 0.1 wt % 4f (3.6 g) and slowly cooled to 0° C. at a rate of 10° C. per hour. Ethanol (200 proof, 5 L) was added to maintain a stirrable mixture. The mixture was slurried at 0° C. for ˜13.5 hours. The solids were filtered and the cake was washed with ethanol (200 proof, 7.2 L) then dried at 50° C. under vacuum to provide 4f (1.1 kg, 20.2% yield, 98.1% chemical purity, 97.9% isomeric purity).

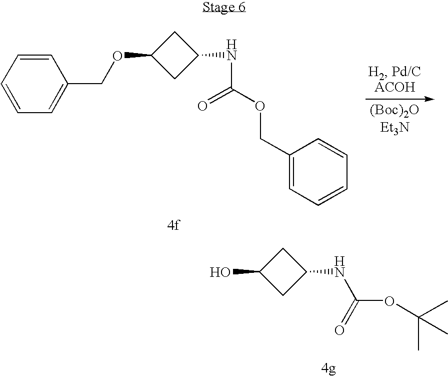

A suspension of 4f (27 g) and Pd/C (3.9 g, 10% w/w) in acetic acid (163 mL) was shaken under ˜50 psi for 1 hour at 20° C. and heated at 50° C. for ˜3-5 hours. The reaction mixture was cooled to room temperature, filtered, washed with ethanol, and the filtrate concentrated until ˜1 volume was left. The residue oil was dissolved in ethanol (55 mL) and triethylamine (55 mL). Di-t-butoxy dicarbonate (14.5 g) was added, and the reaction mixture was stirred at room temperature over the weekend. The solution was concentrated to minimum volume. Water (110 mL) and methylene chloride (68 mL) and then saturated sodium bicarbonate (34 mL) were added. Two layers were separated. The aqueous layer was extracted with methylene chloride (2×68 mL) again. The combined organic layers were washed with brine (33 mL). The solution was then concentrated. Cyclohexane (108 mL) was added and then concentrated to 3.0 volumes. The solid was filtered, washed with cyclohexane (27 ml) and the wet cake was dried under vacuum at 50° C. to provide 15.5 g of product, 4 g, in a yield of 95%.

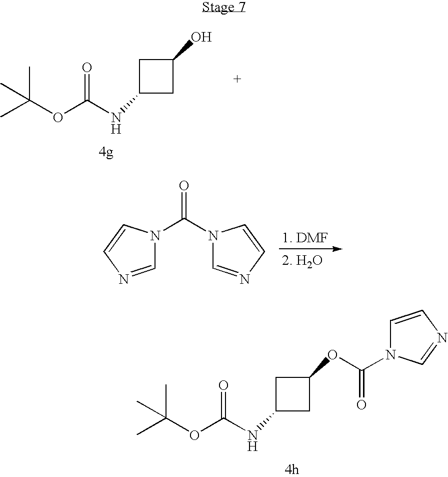

To a solution of 4 g (17.9 g) in N,N-dimethylformamide (DMF) (72 mL) resulting in a clear solution was added 1,1′-carbonyldiimidazole (CDI) (20.2 g, 1.3 equiv) was charged, which caused the internal temperature to rise to ˜30° C. The mixture was stirred at room temperature for ˜30 minutes before sampling and concluding 4 g was consumed based on HPLC results. While the mixture was cooled with ice bath, water (˜160 mL) was added and the mixture was stirred at ˜0° C. for 45 minutes. The solid was filtered and mixed with water (˜80 mL). The solid was dried under vacuum at ˜55° C. overnight and 24 g of product 4 h was obtained.

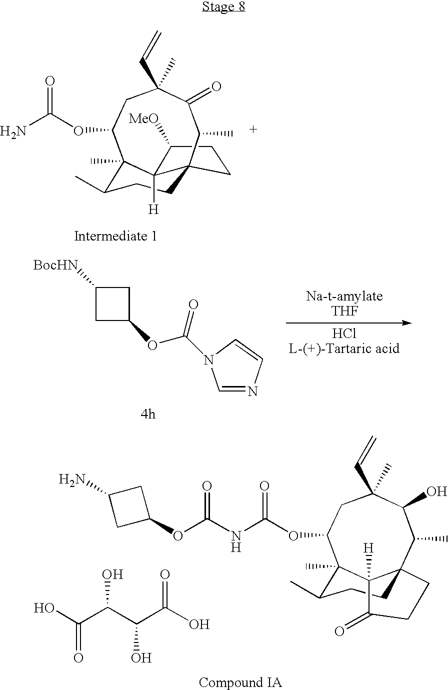

A solution of Intermediate 1 (1.89 g, 5 mmol) in THF (19 mL) was cooled to ˜17° C. Sodium tert-pentoxide (1.38 g, 12.5 mmol, 2.5 eq.) was added in one portion. The solution was stirred for 30 minutes before addition of 4 h (1.54 g, 5.5 mmol, 1.1 eq.) in one portion at ˜18° C. The solution was stirred for one to two hours. Water (9.5 mL) was added slowly to quench the reaction, followed by the addition of saturated ammonium chloride solution (9.5 mL). Ethyl acetate (17 mL) was added and then the mixture was stirred for 5 minutes. The resulting two layers were separated and the aqueous layer was extracted with ethyl acetate (3.8 mL). The combined organic layers were washed with saturated ammonium chloride solution (1.9 mL) then concentrated to ˜15 mL.

Hydrochloric acid (conc. 8.0 mL) was added slowly at room temperature and the resulting solution was stirred at 50° C. for ˜4 hours. The solution was then cooled to ˜15° C. and water (4 mL) was added. Sodium hydroxide solution (25%, 14 mL) was added slowly to bring the pH to ˜13. Ethyl acetate (19 mL) was added and two layers were separated. The aqueous layer was extracted with ethyl acetate (2×10 mL). The combined organic layers were washed with water (4 mL), dried over sodium sulfate and then concentrated to minimum volume. Acetone (24 mL) was added and stirred until a clear solution was obtained. A solution of L-(+)-tartaric acid (0.75 g, 5.0 mmol, 1.0 eq) in water (2.0 mL) was added under stirring. The suspension was then stirred for 1 hour at room temperature before isolation. The solid was washed with acetone (2 mL) and then dried under house vacuum at 50° C. Compound IA as a white solid (2.4 g) was obtained.

### Example 5

### Preparation of intermediate 4f from Example 4

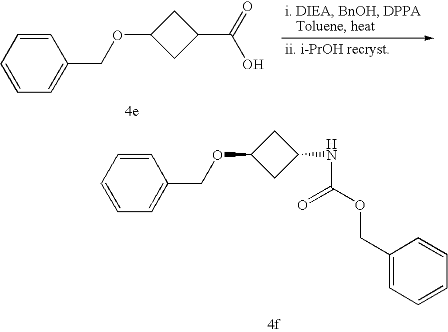

A solution of 4e (22.3 g) in toluene (220 mL) and N,N-diisopropylethylamine (41 mL,) was prepared then heated to an internal temperature between 95 and 105° C. Diphenyl-phosphorylazide (26 mL) was slowly added over 40 minutes at a rate to maintain manageable levels of heat and gas evolution. The mixture was stirred for 10 minutes, then benzyl alcohol (12.5 mL) was added and the reaction was stirred for about 10 hours at a temperature about 95° C. The reaction mixture was cooled to ambient temperature. Diluted with Ethyl acetate (EtOAc, 250 mL) then washed with 0.25 N aqueous sodium hydroxide (NaOH) solution (125 mL). The layers were separated. The organic layer was washed with 0.25 N NaOH (150 mL). EtOAc was removed via rotary evaporation. Isopropanol (100 mL) was added and the mixture was heated to 80° C. to dissolve all solids, then cooled to between 30 and 40° C. The mixture was seeded with 0.1 wt % 4f at an internal temperature of 35° C. and slowly cooled to between 0° C. and 5° C. The mixture was slurried at 0° C. for 1 hour, then filtered and washed with isopropanol (0-10° C.) as needed (˜15 ml). The product was dried at 50° C. under vacuum to provide 4f (13.3 g, 39% yield, 90.6% isomeric purity, 90.2% chemical purity).

### Example 6

### Preparation of Compound IA

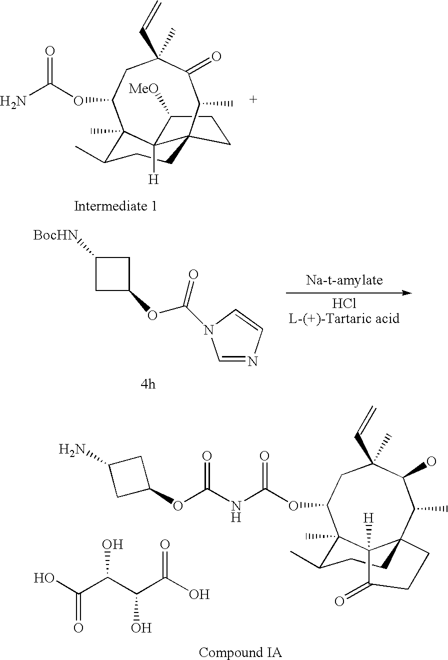

A solution of Intermediate 1 (807 g) in THF (4.0 L) was cooled to ˜0° C. in a 20 L jacketed laboratory reactor. Sodium tert-pentoxide (587 g) was added portionwise over ˜7 minutes to maintain the reaction temperature below 10° C. The solution was warmed to ˜15° C. over ˜15 minutes, then stirred at this temperature for ˜70 minutes, before cooling the solution back down to approximately ˜5 to 0° C. A solution of intermediate 4 h (600 g) in THF (4.0 L) was added slowly over ˜25 minutes at a rate sufficient to keep the reaction temperature below 5° C. The solution was stirred for ˜2 hours. Water (3.2 L) was added slowly to quench the reaction while keeping the reaction temperature at ˜18° C. The solution was concentrated under vacuum until ˜6.5 L of solution remained. Dichloromethane (6.0 L) was added and the resulting two layers were separated and the aqueous layer was extracted twice with dichloromethane (3.0 L each). The combined organic layers were washed with water (1.7 L) then the organic layer was concentrated until ˜2 L of solution remained. THF (2.8 L) was added and the solution was kept at ambient conditions for ˜2.5 days.

The solution was cooled to ˜5° C. and concentrated hydrochloric acid (3.4 L) was added slowly while maintaining the solution temperature at <25° C. The resulting mixture was warmed to 34° C. over 20 minutes and stirred at ˜35° C. for ˜2.5 hours at which point the reaction was deemed complete by HPLC. The solution was then cooled to ˜5° C. over ˜20 minutes and water (1.6 L) was added. Sodium hydroxide solution (25%) was added slowly to bring pH to ˜9.0. The mixture was concentrated under vacuum at ˜15° C. Dichloromethane (6.0 L) was added and the two layers were separated. The aqueous layer was extracted with dichloromethane (3.0 L then 2.0 L). The combined organic layers were washed with brine (2.0 L) and then water (2.0 L) then concentrated to a final volume of ˜2.2 L then held overnight. Acetonitrile (9.6 L) was added and the solution was concentrated under vacuum until ˜12 L remained. Water (580 mL) was added and the mixture was heated to ˜50-55° C. before a solution of L-(+)-tartaric acid (319 g) in water (0.58 L) was added portionwise under stirring. The solution was then stirred for 1 hour at 55° C., a thick slurry being formed, and then cooled down to ˜15° C. over 55 minutes before isolation by filtration. The solid was washed with acetonitrile (1.6 L) and then dried under house vacuum at 50° C. Compound IA as a white solid (966 g) was obtained in a yield of 70%.

### Example 7

### Recrystallization of Compound IA

A 1000 mL jacketed reactor was charged with 50 g of crude Compound IA. Acetonitrile (150 mL) and water (62.5 mL) were charged. The slurry was stirred and heated to 70° C. to give a clear solution. When dissolved the solution was cooled to 65° C. over about 15 minutes, and held for about 1 hour. Ground seed crystals (0.1 w/w %) were added (50 mg in 5 mL acetonitrile). The resulting suspension was stirred for 30 minutes at 65° C. and then cooled to 50° C. over about 20 minutes. Maintaining the temperature at 48-50° C., 55 mL of acetonitrile was charged every 15 minutes for 2 hours. The slurry was cooled to 0° C. over 1.5 hours. The cold slurry was stirred for 15 hours, and the solid was then isolated by filtration under pressure. The reactor and cake were washed with acetonitrile (200 mL). The cake was dried under briefly under nitrogen pressure and heated to 50-55° C. under vacuum for 3 hours. The procedure gave 46.2 g of white solid, Compound IA, 92% by weight.

### Example 8

### Preparation of Protected Compound IA

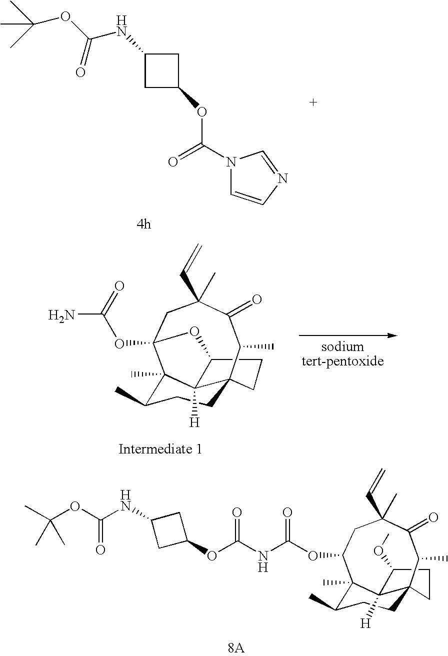

Intermediate 1 (10.0 g) was mixed in toluene (35 mL) and cooled in an ice bath. A 25% wt solution of sodium tert-pentoxide (29.2 g solution) in toluene was added to the mixture, temperature reaches 12° C., and the mixture is stirred for 1 to 2 h. A solution of 4 h (10.0 g) in 20 mL of NMP and 40 mL of toluene was prepared and added to the reaction over 5 minutes. The temperature reaches 9° C. The reaction was stirred overnight and quenched with a 2 M citric acid solution (50 mL), stirred for ½ hour, and a pH of 4-5 was obtained. The phases were separated and the organic layer was washed with water (50 mL). The phases were separated and the organic phase containing the product was stored until further use.

### Example 9

### Preparation of Compound I

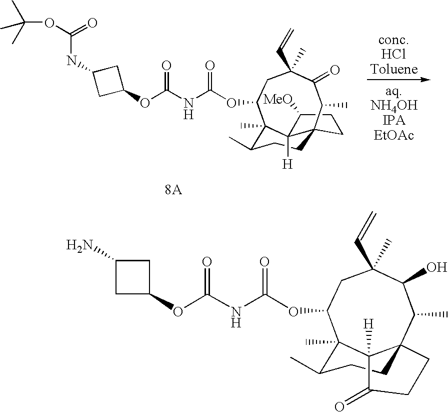

A solution of 8a (8.2 g crude) in toluene (55 ml) was cooled to 15° C. and concentrated HCl (12.3 mL) was added. The mixture was stirred for 3 hours the stored at 0° C. over the weekend. The layers were separated into two phases, and the bottom aqueous layer was slowly added over 5 hours into a second vessel containing a mixture of 30% ammonium hydroxide (16.4 mL), water (8.2 mL), IPA (8.2 mL), and ethyl acetate (32.8 mL) that has been cooled to 15° C. After stirring the mixture at ˜25° C. for 0.5 hours the bottom aqueous layer was separated and discarded and the top organic layer was washed with water (24.6 mL). On concentration of the top layer, 7.5 g of the crude product as an oil was obtained.

### Example 10

### Recrystallization of Compound IA

206 mg of Compound IA was dissolved in 11 volumes of Acetonitrile/water (10/1, vol/vol). The clear solution was cooled and seeded with Compound IA. The white solid was isolated at room temperature to give an 80% yield. The solid was analyzed by HPLC (92.4% PAR), DSC and microscopy.

### Example 11

### Recrystallization of Compound IA

173 mg of Compound IA was dissolved at reflux in 15 volumes of n-propanol containing 3% by volume of water. The solution was cooled to 0° C. to promote crystallization. The white solid was isolated in 55% yield. The solid was analyzed by HPLC (94% PAR), DSC and microscopy.

### Example 12

### Recrystallization of Compound IA

258 mg of Compound IA was dissolved at reflux in 15 volumes of n-propanol containing 5% by volume of water. The solution was cooled to 0° C. to promote crystallization. The white solid was isolated in 76% yield. The solid was analyzed by HPLC (96.9% PAR), and microscopy.

### Example 13

### Recrystallization of Compound IA

151 mg of Compound IA was dissolved at reflux in 15 volumes of 2-butanone containing 7% by volume of water. The solution was seeded, then cooled to 0° C. to promote crystallization. The white solid was isolated in 67% yield. The solid was analyzed by HPLC (88.6% PAR), DSC, and microscopy.

### Example 14

### Recrystallization of Compound IA

1.87 g of Compound IA was charged with 5 volumes of Acetone and 0.25 vol of water and heated to dissolve. The clear solution was cooled to room temperature slowly. After sitting overnight, solids appeared. The suspension was cooled to 0° C. and isolated. Microscopy was done.

### Example 15

### Recrystallization of Compound IA

3 g of Compound IA was dissolved in 5 volumes of Acetonitrile/water (3/1, vol/vol) at 75° C. The clear solution was cooled to 65° C. and seeded with Compound IA. The suspension was diluted with 8 volumes of acetonitrile over 10 minutes. The suspension was cooled to 0° C. and held overnight. The white solid was isolated and dried under vacuum at 55° C. overnight to give 92% yield. Solid was analyzed by XRPD, ¹H NMR, HPLC (93.9% PAR), DSC, TG, LOD (4.25%) and microscopy.

### Example 16

### Preparation of Compound IA

410 mg of Compound I was dissolved in 15 volumes of 2-propanol. A clear solution of tartaric acid dissolved in water (142 mg, 1.1 eq, in 0.31 mL of water) was added. The cloudy suspension was heated to dissolve at reflux. The clear solution was cooled to 50° C. and seeded with Compound IA. An additional 5 volumes of 2-propanol was added to aid stirring. The white solid was isolated at 0° C. to give 78.3% yield. The solid was analyzed by HPLC (95.6% PAR), DSC, TG and microscopy.

### Example 17

### Preparation of Compound IA

525 mg of Compound I was dissolved in 8 volumes of acetonitrile. A clear solution of tartaric acid dissolved in water (165 mg, 1.1 eq, in 0.42 mL of water) was added. The cloudy suspension was heated to dissolve. An additional 2 volumes acetonitrile and 1 volume water was required to dissolve at reflux. The clear solution was cooled to 50° C. and seeded with Compound IA. An additional 5 volumes of acetonitrile was added to aid stirring. The white solid was isolated at 0° C. to give 79.5% yield. The solid was analyzed by EPLC 96.2% PAR), DSC, TG and microscopy.

### Example 18

### Preparation of Compound IA

1 g of Compound I was dissolved in 10 volumes of acetonitrile at a bath temperature of 78° C. A clear solution of tartaric acid dissolved in water (346 mg, 1.1 eq, in 1 mL of water) was added. The contents became clear, then crystallization began spontaneously. An additional 1.5 volumes of water was added to dissolve all solids at reflux. The clear solution was cooled to 70° C. and seeded with 1% by weight of Compound IA. An additional 5 volumes of acetonitrile was added to aid stirring. The white solid (needles) was isolated at 0° C. to give 68.4% yield. The solid was analyzed by DSC, TG and microscopy.

### Methods of Use

Compound I demonstrates good in vitro antibacterial activity against the primary respiratory pathogens including S. pneumoniae, H. influenzae, M. catarrhalis, S. aureus, and S. pyogenes, as well as activity against isolates carrying resistance determinants to other antibiotics (penicillin-, macrolide-, methicillin- or levofloxacin-resistant phenotypes). Compound I also demonstrates good in vitro activity against atypical pathogens including C. pneumoniae, L. pneumophila and M. pneumoniae. Additionally, Compound I demonstrates good in vitro activity against biothreat organism F. tularensis, anaerobic organisms, and Neisserria sp. including N. meningitidis and both ciprofloxacin susceptible and resistant N. gonorrhoeae. Accordingly, in another aspect the invention is directed to methods of treating respiratory infections comprising administering a safe and effective amount of Compound IA to a patient in need thereof.

Compound I demonstrates good in vitro antibacterial activity against S. aureus and S. pyogeizes, the primary pathogens associated with skin and skin structure infections. Activity of Compound I is also retained against S. aureus and S. pyogenes isolates carrying resistance determinants to other antibiotics (penicillin-, macrolide-, methicillin- or levofloxacin-resistant phenotypes). Accordingly, in another aspect the invention is directed to methods of treating skin and skin structure infections comprising administering a safe and effective amount of Compound IA to a patient in need thereof.

Assays for testing the antibacterial activity of Compound IA are known to those skilled in the art.

As used herein, “patient” refers to a human or other animal.

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition.

As indicated above, “treatment” of a condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” in reference to Compound IA or other pharmaceutically-active agent means an amount of the compound sufficient to treat the patient's condition but low enough to avoid serious side effects (at a reasonable benefit/risk ratio) within the scope of sound medical judgment. A safe and effective amount of a compound will vary with the particular compound chosen (e.g. consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for Compound IA depend on the pharmacokinetic properties of the compound, such as absorption, distribution, and half-life, which can be determined by the skilled artisan. In addition, suitable dosing regimens, including the duration such regimens are administered, for Compound IA depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patients response to the dosing regimen or over time as individual patient needs change.

Typical daily dosages may vary depending upon the particular route of administration chosen. Typical daily dosages for oral administration range from about 100 mg to about 3000 mg per day. In one embodiment of the invention, the patient is administered from about 250 mg to about 2000 mg per day. In another embodiment, the patient is administered from about 1000 mg to about 2000 mg per day. In another embodiment, the patient is administered about 1000 mg per day. In another embodiment, the patient is administered about 2000 mg per day.

The invention also provides Compound IA for use in medical therapy, and particularly in respiratory and skin and skin structure infections. Thus, in further aspect, the invention is directed to the use of Compound IA in the preparation of a medicament for the treatment of respiratory and skin and skin structure infections.

### Compositions

The compounds of the invention will normally, but not necessarily, be formulated into pharmaceutical compositions prior to administration to a patient. Accordingly, in another aspect the invention is directed to pharmaceutical compositions comprising Compound IA and one or more pharmaceutically-acceptable excipient.

The pharmaceutical compositions of the invention may be prepared and packaged in bulk form wherein a safe and effective amount of Compound IA can be extracted and then given to the patient such as with powders or syrups. Alternatively, the pharmaceutical compositions of the invention may be prepared and packaged in unit dosage form wherein each physically discrete unit contains a safe and effective amount of Compound IA. When prepared in unit dosage form, the pharmaceutical compositions of the invention typically contain from about 100 mg to about 1000 mg.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the Compound IA when administered to a patient and interactions which would result in pharmaceutical compositions that are not pharmaceutically acceptable are avoided. In addition, each excipient must of course be of sufficiently high purity to render it pharmaceutically-acceptable.

The Compound IA and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; and (3) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of Compound IA once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of Compound IA and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

1. An L-tartrate salt of trans-3-aminocyclobutyl (1S,2R,3S,4S,6R,7R, 8R, 14R)-4-ethenyl-3-hydroxy-2,4,7,14-tetramethyl-9-oxotricyclo[5.4.3.01,8]tetradec-6-yl imidodicarbonate.

2. The salt according to claim 1 wherein the salt is represented by the following structure:

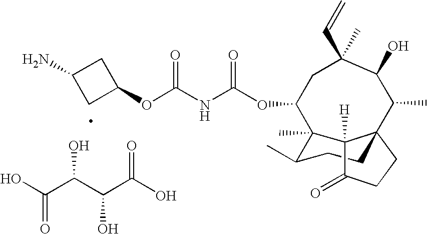

3. The salt according to claim 2 in the solid-state.

4. The salt according to claim 1 wherein the salt is a solvate.

5. The salt according to claim 4 wherein the salt is a non-stoichiometric hydrate.

6. The non-stoichiometric hydrate according to claim 5 wherein the salt contains from about 2% to about 7% water.

7. The non-stoichiometric hydrate according to claim 5 wherein the salt contains from about 2% to about 6% water.

8. The non-stoichiometric hydrate according to claim 5 wherein the salt contains from about 4% to about 6% water.

9. The salt according to claim 1 wherein the salt is in crystalline form.

10. The salt according to claim 1 wherein the salt is characterized by an XRPD pattern that is substantially the same as the XRPD pattern depicted in FIG. 1.

11. A pharmaceutical composition comprising the salt according to claim 1 and one or more pharmaceutically-acceptable excipient.

12-13. (canceled)

14. The salt according to claim 9 which is characterized by an XRPD pattern having characteristic peaks at the following positions: 6.7±0.1 (° 2θ), 10.0±0.1 (° 2θ), 11.7±0.1 (° 2θ), 13.2±0.1 (° 2θ), 13.7±0.1 (° 2θ), 14.2±0.1 (° 2θ), 20.4±0.1 (° 2θ), and 23.5±0.1 (° 2θ).
